(19) United States
(12) Patent Application Publication
(10) Pub. No.: US 2024/0189049 A1
(43) Pub. Date: Jun. 13, 2024

(54) SYSTEMS AND METHODS FOR POINT OF INTERACTION DISPLAYS IN A TELEOPERATIONAL ASSEMBLY

(71) Applicant: Intuitive Surgical Operations, Inc., Sunnyvale, CA (US)

(72) Inventors: Brandon D. Itkowitz, San Jose, CA (US); Timothy B. Hulford, San Jose, CA (US); Tabish Mustufa, Sunnyvale, CA (US); Manqi Xu, Palo Alto, CA (US)

(21) Appl. No.: 18/537,354

(22) Filed: Dec. 12, 2023

Related U.S. Application Data
(63) Continuation of application No. 16/637,926, filed on Feb. 10, 2020, now abandoned.
(60) Provisional application No. 62/543,594, filed on Aug. 10, 2017.

(51) Int. Cl. A61B 34/35; A61B 17/00; A61B 34/00; A61B 34/20; A61B 90/00

(52) U.S. Cl. CPC A61B 34/35; A61B 34/20; A61B 2017/00973; A61B 2034/2051; A61B 2034/2055; A61B 2034/256; A61B 2034/741; A61B 2034/742; A61B 2090/366; A61B 2217/005; A61B 2217/007

(57) ABSTRACT

A teleoperational system in a surgical environment is provided. The system comprises a teleoperational assembly including a first teleoperational arm and a visual projection device coupled to the teleoperational assembly. The system further comprises a sensor and one or more processors. The one or more processors are configured to: receive first sensor information from the sensor; determine a first visual aid based upon the first sensor information; operate the visual projection device to project the first visual aid into the surgical environment; and operate the visual projection device to change the first visual aid to a second visual aid based on second sensor information received from the sensor.

### CROSS-REFERENCE TO RELATED APPLICATIONS

This application claims the benefit of U.S. Provisional Application 62/543,594 filed Aug. 10, 2017, which is incorporated by reference herein in its entirety.

### FIELD

The present disclosure is directed to surgical systems and methods for performing minimally invasive teleoperational medical procedures using minimally invasive medical techniques, and more particularly to systems and methods for providing point of interaction displays for use by operating room clinicians during medical procedures.

### BACKGROUND

Minimally invasive medical techniques are intended to reduce the amount of extraneous tissue that is damaged during diagnostic or surgical procedures, thereby reducing patient recovery time, discomfort, and harmful side effects. Such minimally invasive techniques may be performed through natural orifices in a patient anatomy or through one or more surgical incisions. Through these natural orifices or incisions, clinicians may insert medical tools to reach a target tissue location. Minimally invasive medical tools include instruments such as therapeutic instruments, diagnostic instruments, and surgical instruments. Minimally invasive medical tools may also include imaging instruments such as endoscopic instruments that provide a user with a field of view within the patient anatomy.

Some minimally invasive medical tools may be teleoperated or otherwise computer-assisted. A clinician near a teleoperational system may need to receive guidance in the form of instructions, warnings, confirmations, or the like either before, during, or after a medical procedure performed with the teleoperational system. Systems and method for providing a point of interaction visual display of guidance information are needed.

### SUMMARY

The embodiments of the invention are summarized by the claims that follow below. In an embodiment, a teleoperational system in a surgical environment comprises a teleoperational assembly including a first teleoperational arm, a first display device coupled to the teleoperational assembly, and a processor. The processor is configured to monitor a location of the first display device in the surgical environment and render a first image on the on the first display device. The first image is rendered based upon the location of the first display device in the surgical environment.

In another embodiment, a method comprises monitoring a location of a first display device in a surgical environment and rendering a first image on the first display device based upon the location of the first display device in the surgical environment.

In another embodiment, a teleoperational system in a surgical environment comprises a teleoperational assembly including a first teleoperational arm a visual projection device coupled to the teleoperational assembly, a sensor, and a processor. The processor is configured to receive first sensor information from the sensor, determine a first visual aid based upon the first sensor information, operate the visual projection device to project the first visual aid into the surgical environment, and operate the visual projection device to change the first visual aid to a second visual aid based on second sensor information received from the sensor.

In another embodiment, a method comprises receiving first sensor information from a sensor of a teleoperational system in a surgical environment, determining a first visual aid based upon the first sensor information, and operating a visual projection device to project the first visual aid into the surgical environment. The visual projection device is coupled to a teleoperational assembly of the teleoperational system. The method also comprises receiving second sensor information from the sensor, determining a second visual aid based upon the second sensor information, and operating the visual projection device to change the first visual aid to the second visual aid.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In addition, the present disclosure may repeat reference numerals and/or letters in the various examples. This repetition is for the purpose of simplicity and clarity and does not in itself dictate a relationship between the various embodiments and/or configurations discussed.

FIG. 1A is a schematic view of a teleoperational medical system, in accordance with an embodiment of the present disclosure.

FIG. 1B is a perspective view of a teleoperational assembly, according to one example of principles described herein.

FIG. 1C is a perspective view of a surgeon's control console for a teleoperational medical system, in accordance with an embodiment.

FIG. 2 illustrates a method of providing information in a surgical environment according to an embodiment of the disclosure.

FIG. 3 illustrates another method of providing information in a surgical environment according to an embodiment of the disclosure.

FIG. 4 illustrates a surgical environment in which a visual aid is used to assist initial patient approach.

FIG. 5 illustrates a surgical environment in which a visual aid is used to assist positioning of an orienting platform.

FIG. 6 illustrates a surgical environment in which a visual aid is used to assist with orientation of the orienting platform.

FIG. 7 illustrates a surgical environment in which another visual aid is used to assist with orientation of the orienting platform.

FIG. 8 illustrates a surgical environment in which a visual aid is used to highlight a portion of the teleoperational assembly.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. In the following detailed description of the aspects of the invention, numerous specific details are set forth in order to provide a thorough understanding of the disclosed embodiments. However, as would be appreciated by one skilled in the art, embodiments of this disclosure may be practiced without these specific details. In other instances well known methods, procedures, components, and circuits have not been described in detail so as not to unnecessarily obscure aspects of the embodiments of the invention.

Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative embodiment can be used or omitted as applicable from other illustrative embodiments. For the sake of brevity, the numerous iterations of these combinations will not be described separately. For simplicity, in some instances the same reference numbers are used throughout the drawings to refer to the same or like parts.

The embodiments below will describe various instruments and portions of instruments in terms of their state in three-dimensional space. As used herein, the term “position” refers to the location of an object or a portion of an object in a three-dimensional space (e.g., three degrees of translational freedom along Cartesian X, Y, Z coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom).

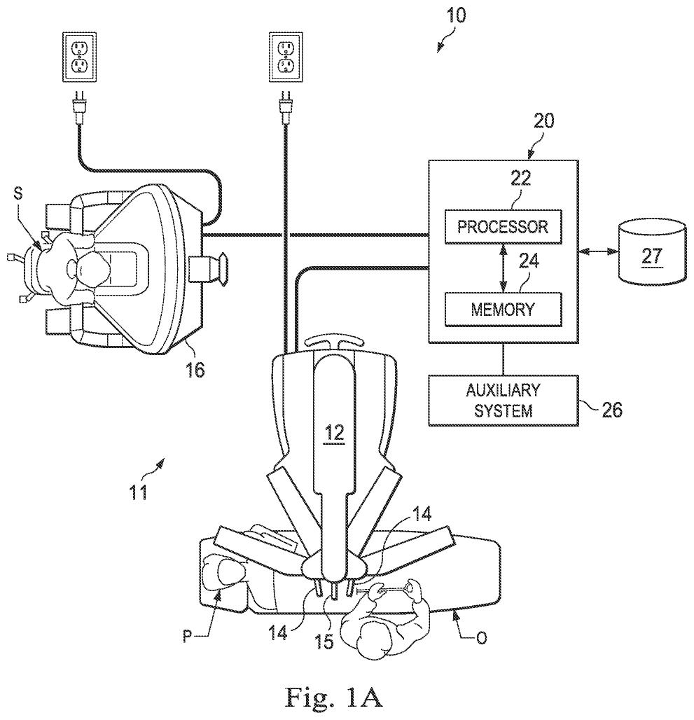

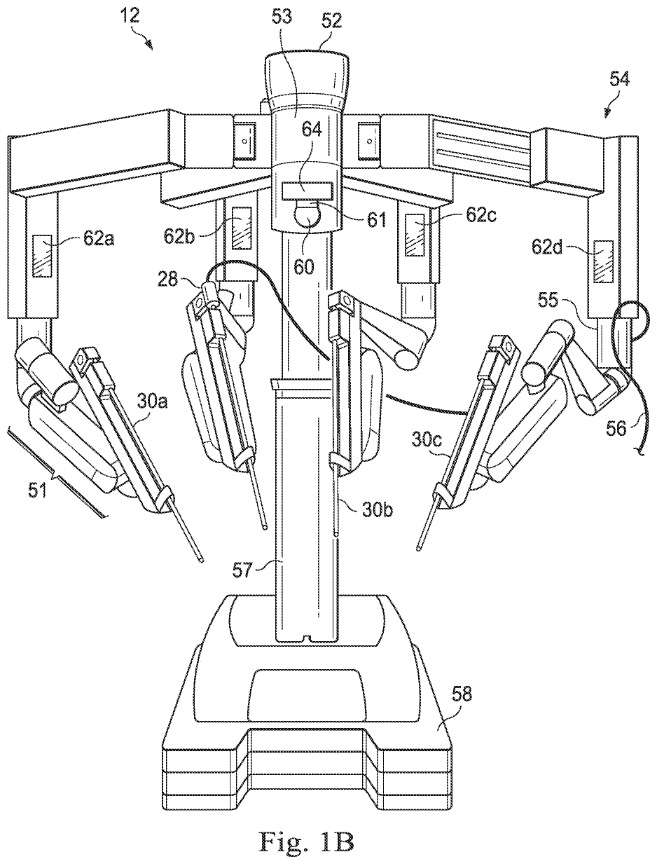

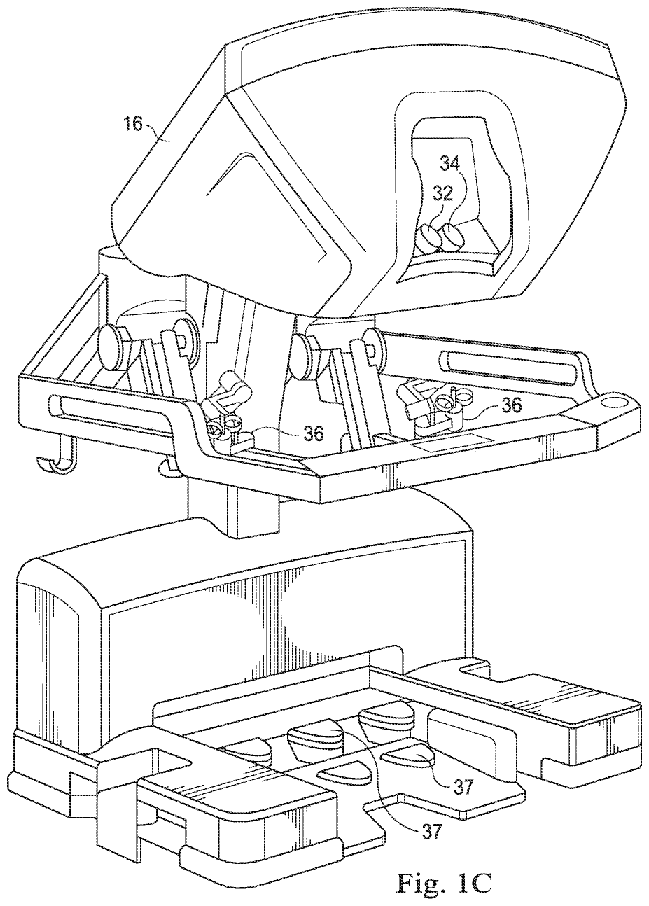

Referring to FIG. 1A of the drawings, a teleoperational medical system for use in, for example, medical procedures including diagnostic, therapeutic, or surgical procedures, is generally indicated by the reference numeral 10. The system 20 is located in the surgical environment 11As will be described, the teleoperational medical systems of this disclosure are under the teleoperational control of a surgeon. In alternative embodiments, a teleoperational medical system may be under the partial control of a computer programmed to perform the procedure or sub-procedure. In still other alternative embodiments, a fully automated medical system, under the full control of a computer programmed to perform the procedure or sub-procedure, may be used to perform procedures or sub-procedures. One example of a teleoperational medical system that may be used to implement the systems and techniques described in this disclosure is the da Vinci® Surgical System manufactured by Intuitive Surgical, Inc. of Sunnyvale, California.

As shown in FIG. 1A, the teleoperational medical system 10 generally includes a teleoperational assembly 12 which may be mounted to or positioned near an operating table O on which a patient P is positioned. The teleoperational assembly 12 may be referred to as a patient side cart, a surgical cart, teleoperational arm cart, or a surgical robot. A medical instrument system 14 and an endoscopic imaging system 15 are operably coupled to the teleoperational assembly 12. An operator input system 16 allows a surgeon or other type of clinician S to view images of or representing the surgical site and to control the operation of the medical instrument system 14 and/or the endoscopic imaging system 15. It should be understood that the medical instrument system 14 may comprise one or more medical instruments. In embodiments in which the medical instrument system 14 comprises a plurality of medical instruments, the plurality of medical instruments may include multiple of the same medical instrument and/or multiple different medical instruments. Similarly, the endoscopic imaging system 15 may comprise one or more endoscopes. In the case of a plurality of endoscopes, the plurality of endoscopes may include multiple of the same endoscope and/or multiple different endoscopes.

The operator input system 16 may comprise a surgeon's console and may be located in the same room as operating table O. It should be understood, however, that the surgeon S and operator input system 16 can be located in a different room or a completely different building from the patient P. Operator input system 16 generally includes one or more control device(s) for controlling the medical instrument system 14. The control device(s) may include one or more of any number of a variety of input devices, such as hand grips, joysticks, trackballs, data gloves, trigger-guns, foot pedals, hand-operated controllers, voice recognition devices, touch screens, body motion or presence sensors, and the like. In some embodiments, the control device(s) will be provided with the same degrees of freedom as the medical instruments of the medical instrument system 14 to provide the surgeon with telepresence, the perception that the control device(s) are integral with the instruments so that the surgeon has a strong sense of directly controlling instruments as if present at the surgical site. In other embodiments, the control device(s) may have more or fewer degrees of freedom than the associated medical instruments and still provide the surgeon with telepresence. In some embodiments, the control device(s) are manual input devices which move with six degrees of freedom, and which may also include an actuatable handle for actuating instruments (for example, for closing grasping jaw end effectors, applying an electrical potential to an electrode, delivering a medicinal treatment, and the like).

The teleoperational assembly 12 supports and manipulates the medical instrument system 14 while the surgeon S views the surgical site through the operator input system 16. An image of the surgical site can be obtained by the endoscopic imaging system 15, which can be manipulated by the teleoperational assembly 12. The teleoperational assembly 12 may comprise endoscopic imaging systems 15 and may similarly comprise multiple medical instrument systems 14 as well. The number of medical instrument systems 14 used at one time will generally depend on the diagnostic or surgical procedure to be performed and on space constraints within the operating room among other factors. The teleoperational assembly 12 may include a kinematic structure of one or more non-servo controlled links (e.g., one or more links that may be manually positioned and locked in place, generally referred to as a set-up structure) and a teleoperational manipulator. The teleoperational assembly 12 includes a plurality of motors that drive inputs on the medical instrument system 14. In an embodiment, these motors move in response to commands from a control system (e.g., control system 20). The motors include drive systems which when coupled to the medical instrument system 14 may advance a medical instrument into a naturally or surgically created anatomical orifice. Other motorized drive systems may move the distal end of said medical instrument in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the motors can be used to actuate an articulable end effector of the medical instrument for grasping tissue in the jaws of a biopsy device or the like. Medical instruments of the medical instrument system 14 may include end effectors having a single working member such as a scalpel, a blunt blade, an optical fiber, or an electrode. Other end effectors may include, for example, forceps, graspers, scissors, or clip appliers.

The teleoperational medical system 10 also includes a control system 20. The control system 20 includes at least one memory 24 and at least one processor 22 for effecting control between the medical instrument system 14, the operator input system 16, and other auxiliary systems 26 which may include, for example, imaging systems, audio systems, fluid delivery systems, display systems, illumination systems, steering control systems, irrigation systems, and/or suction systems. A clinician C may circulate within the surgical environment 11 and may access, for example, the assembly 12 during a set up procedure or view a display of the auxiliary system 26 from the patient bedside.

Though depicted as being external to the teleoperational assembly 12 in FIG. 1A, the control system 20 may, in some embodiments, be contained wholly within the teleoperational assembly 12. The control system 20 also includes programmed instructions (e.g., stored on a non-transitory, computer-readable medium) to implement some or all of the methods described in accordance with aspects disclosed herein. While control system 20 is shown as a single block in the simplified schematic of FIG. 1A, the system may include two or more data processing circuits with one portion of the processing optionally being performed on or adjacent the teleoperational assembly 12, another portion of the processing being performed at the operator input system 16, and the like. Any of a wide variety of centralized or distributed data processing architectures may be employed. Similarly, the programmed instructions may be implemented as a number of separate programs or subroutines, or they may be integrated into a number of other aspects of the teleoperational systems described herein. In one embodiment, control system 20 supports wireless communication protocols such as Bluetooth, IrDA, HomeRF, IEEE 802.11, DECT, and Wireless Telemetry.

The control system 20 is in communication with a database 27 which may store one or more clinician profiles, a list of patients and patient profiles, a list of procedures to be performed on said patients, a list of clinicians scheduled to perform said procedures, other information, or combinations thereof. A clinician profile may comprise information about a clinician, including how long the clinician has worked in the medical field, the level of education attained by the clinician, the level of experience the clinician has with the teleoperational medical system 10 (or similar systems), or any combination thereof.

The database 27 may be stored in the memory 24 and may be dynamically updated. Additionally or alternatively, the database 27 may be stored on a device such as a server or a portable storage device that is accessible by the control system 20 via an internal network (e.g., a secured network of a medical facility or a teleoperational system provider) or an external network (e.g, the Internet). The database 27 may be distributed throughout two or more locations. For example, the database 27 may be present on multiple devices which may include the devices of different entities and/or a cloud server. Additionally or alternatively, the database 27 may be stored on a portable user-assigned device such as a computer, a mobile device, a smart phone, a laptop, an electronic badge, a tablet, a pager, and other similar user devices.

In some embodiments, control system 20 may include one or more servo controllers that receive force and/or torque feedback from the medical instrument system 14. Responsive to the feedback, the servo controllers transmit signals to the operator input system 16. The servo controller(s) may also transmit signals instructing teleoperational assembly 12 to move the medical instrument system(s) 14 and/or endoscopic imaging system 15 which extend into an internal surgical site within the patient body via openings in the body. Any suitable conventional or specialized servo controller may be used. A servo controller may be separate from, or integrated with, teleoperational assembly 12. In some embodiments, the servo controller and teleoperational assembly 12 are provided as part of a teleoperational arm cart positioned adjacent to the patient's body.

The control system 20 can be coupled with the endoscope 15 and can include a processor to process captured images for subsequent display, such as to a surgeon on the surgeon's console, or on another suitable display located locally and/or remotely. For example, where a stereoscopic endoscope is used, the control system 20 can process the captured images to present the surgeon with coordinated stereo images of the surgical site. Such coordination can include alignment between the opposing images and can include adjusting the stereo working distance of the stereoscopic endoscope.

In alternative embodiments, the teleoperational medical system 10 may include more than one teleoperational assembly 12 and/or more than one operator input system 16. The exact number of teleoperational assemblies 12 will depend on the surgical procedure and the space constraints within the operating room, among other factors. The operator input systems 16 may be collocated, or they may be positioned in separate locations. Multiple operator input systems 16 allow more than one operator to control one or more teleoperational assemblies 12 in various combinations.

FIG. 1B is a perspective view of one embodiment of a teleoperational assembly 12 which may be referred to as a patient side cart, surgical cart, teleoperational arm cart, or surgical robot. The teleoperational assembly 12 shown provides for the manipulation of three surgical tools 30a, 30b, 30c (e.g., medical instrument systems 14) and an imaging device 28 (e.g., endoscopic imaging system 15), such as a stereoscopic endoscope used for the capture of images of the site of the procedure. The imaging device may transmit signals over a cable 56 to the control system 20. Manipulation is provided by teleoperative mechanisms having a number of joints. The imaging device 28 and the surgical tools 30a-c can be positioned and manipulated through incisions in the patient so that a kinematic remote center is maintained at the incision to minimize the size of the incision. Images of the surgical site can include images of the distal ends of the surgical tools 30a-c when they are positioned within the field-of-view of the imaging device 28.

The teleoperational assembly 12 includes a drivable base 58. The drivable base 58 is connected to a telescoping column 57, which allows for adjustment of the height of arms 54. The arms 54 may include a rotating joint 55 that both rotates and moves up and down. Each of the arms 54 may be connected to an orienting platform 53. The arms 54 may be labeled to facilitate trouble shooting. For example, each of the arms 54 may be emblazoned with a different number, letter, symbol, other identifier, or combinations thereof. In FIG. 1B, the arms 54 are numbered from one to four. The orienting platform 53 may be capable of 360 degrees of rotation. The teleoperational assembly 12 may also include a telescoping horizontal cantilever 52 for moving the orienting platform 53 in a horizontal direction.

In the present example, each of the arms 54 connects to a manipulator arm 51. The manipulator arms 51 may connect directly to a medical instrument, e.g., one of the surgical tools 30a-c. The manipulator arms 51 may be teleoperatable. In some examples, the arms 54 connecting to the orienting platform 53 may not be teleoperatable. Rather, such arms 54 may be positioned as desired before the surgeon S begins operation with the teleoperative components. Throughout a surgical procedure, medical instruments may be removed and replaced with other instruments such that instrument to arm associations may change during the procedure. Displays such as displays 62a-d may help reinforce the operative function of each arm based on the currently attached instrument.

Endoscopic imaging systems (e.g., endoscopic imaging system 15 and imaging device 28) may be provided in a variety of configurations including rigid or flexible endoscopes. Rigid endoscopes include a rigid tube housing a relay lens system for transmitting an image from a distal end to a proximal end of the endoscope. Flexible endoscopes transmit images using one or more flexible optical fibers. Digital image based endoscopes have a “chip on the tip” design in which a distal digital sensor such as a one or more charge-coupled device (CCD) or a complementary metal oxide semiconductor (CMOS) device store image data. Endoscopic imaging systems may provide two- or three-dimensional images to the viewer. Two-dimensional images may provide limited depth perception. Three-dimensional stereo endoscopic images may provide the viewer with more accurate depth perception. Stereo endoscopic instruments employ stereo cameras to capture stereo images of the patient anatomy. An endoscopic instrument may be a fully sterilizable assembly with the endoscope cable, handle and shaft all rigidly coupled and hermetically sealed.

A projector 60 (e.g. a type of auxiliary system 26) may be coupled to or integrated into the teleoperational assembly 12. As shown, the projector 60 may be located on the orienting platform 53. In an embodiment, the projector 60 may be centrally located on the underside of the orienting platform 53. In some cases, the projector 60 may be located elsewhere. For example, the projector 60 may be located on one of the arms 54, on the telescoping column 57, on the drivable base 58, on the telescoping horizontal cantilever 52, or elsewhere. The location of the projector 60 may be chosen based at least in part on the kinematics of the teleoperational assembly 12. The projector 60 may be rigidly mounted or integrated with the teleoperational assembly such that the projector is in a known configuration with respect to the kinematically tracked manipulator arms 51. The projector 60 may be located such that movement of manipulator arms 51 during surgery does not change the orientation of the projector 60. Though unaffected by movement of manipulator arms 51, the projector 60 may itself be able to rotate, swivel, pivot, or otherwise move such that images may be projected in different directions without changing the orientation of the teleoperational assembly 12. Only a single projector 60 is depicted in FIG. 1B; however, the teleoperational assembly 12 may comprise multiple projectors 60, e.g., one or more projectors 60 on each of the arms 54.

The projector 60 may be sized and shaped to be housed substantially within or on a component of the teleoperational assembly 12, e.g., arms 54, orienting platform 53, drivable base 58, telescoping column 57, telescoping horizontal cantilever 52, etc. The projector 60 may comprise a Digital Light Processing (DLP), Liquid Crystal on Silicon (LCoS), or Laser Beam Steering (LBS) pico projector or other type of still or moving visual image projector. The projector 60 may project images in color. To minimize the effect of ambient light on images produced by projectors, the projector 60 may produce an image bright enough to be readily perceived despite any ambient light present in the operating room, if any. The projector 60 may project an image having a brightness of about 500 lumens, about 1,000 lumens, about 1,500 lumens, about 2,000 lumens, about 2,500 lumens, about 3,000 lumens, about 3,500 lumens, about 4,000 lumens, about 4,500 lumens, about 5,000 lumens, about 5,500 lumens, about 6,000 lumens, about 6,500 lumens, about 7,000 lumens, or having some other brightness.

The projector 60 may be controlled by the teleoperational assembly 12 and/or by the operator input system 16. In an embodiment, the teleoperational assembly 12 may operate the projector 60 to provide guidance to clinicians in the operating room in the form of visual aids, which may also be accompanied by audible aids. The visual aids may include graphical indicators, symbols, alphanumeric content, light patterns, or any other visual information.

A sensor 61 may be located on the orienting platform 53 or elsewhere and may be used to determine whether or not a patient, or operating table, is positioned within a work zone of the teleoperational assembly 12. For the purposes of this disclosure, the work zone of the teleoperational assembly 12 may be defined by the range of motion of the surgical tools 30a-c. The sensor 61 may be, for example, a depth sensor or a thermal sensor. A thermal sensor may include an infrared sensor that generates sensor information used to determine whether or not a patient is within the work zone by comparing readings from the sensor to an expected thermal profile for a patient. A depth sensor may be, for example, an ultrasonic range finder, an infrared range finder, a laser range finder, a depth camera, or combinations thereof. A depth sensor may measure the distance between the sensor and a surface directly below the sensor. When the teleoperational assembly 12 is not positioned adjacent to an operating table, the surface directly below the sensor may be the floor of the operating room. By contrast, when the teleoperational assembly 12 is positioned adjacent to an operating table, the nearest surface directly below the sensor may be the operating table or a patient positioned on the operating table.

A predetermined distance value may be associated with the height of an operating table. If the sensor information from the depth sensor is greater than the predetermined distance, the sensor information indicates the absence of an operating table and/or patient in the work zone. If the sensor information from the depth sensor is at or less than the predetermined distance, the sensor information indicates the presence of an operating table and/or patient in the work zone. The predetermined distance value may be any value between about 30″ and 60″. For example, the predetermined threshold value may be about 36″, about 40″, about 48″, about 52″, or some other value. In some cases, a second distance value may be set such that the teleoperational assembly 12 determines that it is adjacent to an operating table and/or has a patient positioned within the work zone when the distance between the sensor and the surface directly below the sensor is falls between the two predetermined values.

One or more displays 62a, 62b, 62c, 62d (e.g. a type of auxiliary system 26) may be coupled to or integrated into the teleoperational assembly 12. As discussed in greater detail below, the displays 62a-d may display visual aids including, for example the status of the arms 54 and may serve as an interface allowing clinicians (e.g. clinician C) to receive guidance from and/or issue instructions to the teleoperational assembly 12, among other things. It is contemplated that, in some circumstances, such as an equipment failure on one of the arms 54, the displays 62a-d and the projector 60 may work together to maximize the likelihood that information will be communicated to an operating room clinician. As shown, the teleoperational assembly 12 comprises displays 62a-d with individual displays located on individual arms 54. The teleoperational assembly 12 is depicted as comprising four displays; however, the teleoperational assembly may include more or fewer displays 62. Though displays 62a-d are shown as being located on vertical sections of arms 54, it is contemplated that one or more of displays 62a-d may be located on other sections, e.g., horizontal sections, of the arms 54. Displays 62 may be located such that their positions remain constant or relatively constant relative to the assembly 12 during surgery. Additionally, displays 62 may be located on portions of arms 54 that do not move or experience little movement after an initial set-up procedure.

Displays 62a-d may be located at approximately the eye-level of the clinician C or between about 48 inches and 72 inches above the floor of the surgical environment. Locating the displays 62 at approximately eye-level may improve visibility of the displays 62a-d and may increase the likelihood that information presented on the displays 62a-d will be seen by operating room clinicians.

Displays 62a-d may be sized for location on the arms 54 and may be sized to be accessible around or through a sterile drape. For example, displays 62a-d may be, for example, square or rectangular in shape with dimensions between approximately 5″ and approximately 9″ on a side. In various embodiments, the displays 62a-d are integral to the teleoperational assembly 12 and are in wired communication with other components, e.g., control system 20, of the teleoperational assembly 12. In other embodiments, the displays 62a-d may be removeably attached to the arms 54 of the teleoperational assembly 12 and may be in wireless communication with other components of the teleoperational assembly 12. The displays 62a-d may support a variety of wireless protocols, including Bluetooth, IrDA, HomeRF, IEEE 802.11, DECT, and Wireless Telemetry. The displays 62a-d may be able to communicate with other auxiliary system 26, including cameras and other displays. The displays 62a-d may display images from the endoscopic imaging system 15, from other cameras in the operating room or elsewhere, from other displays in the operating room or elsewhere, or from combinations thereof.

In various embodiments, each display 62a-d is configured to display information regarding the arm 54 on which it is located. For example, in FIG. 1B, display 62a may be configured to display information regarding the arm 54 labeled with the number one. As discussed above, the teleoperational assembly 12 may monitor the condition of its various components. The displays 62a-d may preserve their spatial association with their respective arms by being affixed to the arms or by being mounted to the teleoperational assembly 12 such that content on the display arranged spatially in the the same spatial sequence as the arms, as viewed by a user.

The teleoperational assembly 12 also includes a dashboard 64. As discussed in greater detail below, the dashboard 64 may display the status of the arms 54 and may serve as an interface allowing clinicians to issue instructions to the teleoperational assembly 12, among other things. It is contemplated that, in some circumstances, such as an equipment failure on one of the arms 54, the dashboard 64, displays 62a-d, the projector 60, or combinations thereof may work together to maximize the likelihood that information will be communicated to an operating room clinician. As shown, the teleoperational assembly 12 comprises a single dashboard 64; however, the teleoperational assembly may include a plurality of dashboards 64. Though dashboard 64 is shown as being located the orienting platform 53, it is contemplated that dashboard 64 may be located elsewhere and may be separate from the teleoperational assembly 12. Dashboard 64 may be located such that its position remains constant or relatively constant during surgery. In other words, dashboard 64 may be located on a portion of the teleoperational assembly 12 that does not move or experiences little movement during surgery. The content on the dashboard 64 may be arranged spatially in the the same spatial sequence as the arms, as viewed by a user.

As discussed above with reference to displays 62a-d, dashboard 64 may be located at approximately eye-level or above eye-level during surgery. Similar to the discussion above with reference to displays 62a-d, dashboard 64 may be sized to for location on the orienting platform 53 and may be sized to be accessible around or through a sterile drape. Dashboard 64 may be larger than displays 62. Accordingly, dashboard 64 may be approximately square or rectangular in shape with dimensions between approximately 5″ and approximately 15″ on a side.

In various embodiments, the dashboard 64 is integral to the teleoperational assembly 12 and is in wired communication with other components, e.g., control system 20, of the teleoperational assembly 12. In other embodiments, the dashboard 64 may be superficially attached to the arms the teleoperational assembly 12 and may be in wireless communication with components of the teleoperational assembly 12. The dashboard 64 may support a variety of wireless protocols, including Bluetooth, IrDA, HomeRF, IEEE 802.11, DECT, and Wireless Telemetry. The dashboard 64 may be able to communicate with auxiliary system 26, including cameras and other displays. The dashboard 64 may display images from the endoscopic imaging system 15, from other cameras in the operating room or elsewhere, from displays 62 (and vice versa), from other displays in the operating room or elsewhere, or from combinations thereof.

In an embodiment, the dashboard 64 is configured to display the status of each of the arms 54 and/or the displays 62a-d. The dashboard 64 may display the status of the arms 54 simultaneously or may cycle through them. The cycle may be such that the status of one arm 54 is displayed at a time or such that the status of multiple arms 54 is displayed at a time. When the status of multiple arms 54 is displayed, the cycle may be such that the status of one arm 54 at a time is removed from the screen and replaced by the status of another, or that multiple are removed and replaced at a time. In order to display the status of multiple arms 54, the screen of the dashboard 64 may be divided into sections such that one section displays one status. The sections may be divided by partitions running vertically, horizontally, or both. Clear spatial association to the arms minimizes the likelihood of a user misassociating status/prompts with the wrong manipulator or instrument.

As discussed above, the teleoperational assembly 12 may monitor the condition of its various components. If the teleoperational assembly 12 discovers a problem with one or more of the arms 54, the medical instrument systems 14, the endoscopic imaging system 15, and/or with other components or combinations thereof, the teleoperational assembly 12 may operate the dashboard 64 to display information configured to facilitate resolution of said problems. Dashboard 64 may display a warning image, a diagnosis of the problem, a suggested resolution of the problem, written instructions for identifying and/or resolving the problem, an animation depicting the location and/or resolution of the problem, or some combination thereof.

In addition to identifying problems with arms 54, the dashboard 64 and/or arms 62a-d may display other information regarding the status of the arms 54. The dashboard 64 and/or arms 62a-d may be dynamically updated as the teleoperational assembly 12 continuously monitors the status of the arms 54. The dashboard 64 and/or arms 62a-d may display information about which tools or instruments are located on the arms 54, the number of times said tools or instruments have been used, the expiration date of said tools or instruments, other information, or combinations thereof. The dashboard 64 and/or arms 62a-d may display images of tools or instruments, animations of tools or instruments, descriptions of tools or instruments, usage graphs, usage timelines, other images, or combinations thereof. The dashboard 64 may also provide higher-level system status and prompts pertaining to the operation of the orienting platform or other commonly connected components of the manipulator 12.

The dashboard 64 may serve as an interface allowing clinicians to issue instructions to the teleoperational assembly 12. In an embodiment, the dashboard 64 may feature either a capacitive or resistive touch screen and may comprise a Graphical User Interface (GUI). In some cases, a resistive touch screen may be desired. For example, a resistive touch screen may help preserve sterility by allowing a clinician to interact with the touch screen through use of a stylus or other instrument without having to touch the screen directly, which may ruin the sterility of the clinician's hand. The dashboard 64 may provide clinicians with the same interactive capabilities as those provided by the devices 62. The dashboard 64 may be configured to permit clinicians to issue instructions to any and all of the arms 54 as opposed to just one as may be the case with displays 62.

FIG. 1C is a perspective view of an embodiment of the operator input system 16, which may be referred to as a surgeon's console. The operator input system 16 includes a left eye display 32 and a right eye display 34 for presenting the surgeon S with a coordinated stereo view of the surgical environment that enables depth perception. The operator input system 16 further includes one or more input control devices 36, which in turn cause the teleoperational assembly 12 to manipulate one or more instruments of the endoscopic imaging system 15 and/or medical instrument system 14. The input control devices 36 can provide the same degrees of freedom as their associated instruments to provide the surgeon S with telepresence, or the perception that the input control devices 36 are integral with said instruments so that the surgeon has a strong sense of directly controlling the instruments. To this end, position, force, and tactile feedback sensors (not shown) may be employed to transmit position, force, and tactile sensations from the medical instruments, e.g., surgical tools 30a-c, or imaging device 28, back to the surgeon's hands through the input control devices 36. Input control devices 37 are foot pedals that receive input from a user's foot. Aspects of the operator input system 16, the teleoperational assembly 12, and the auxiliary systems 26 may be adjustable and customizable to meet the physical needs, skill level, or preferences of the surgeon S.

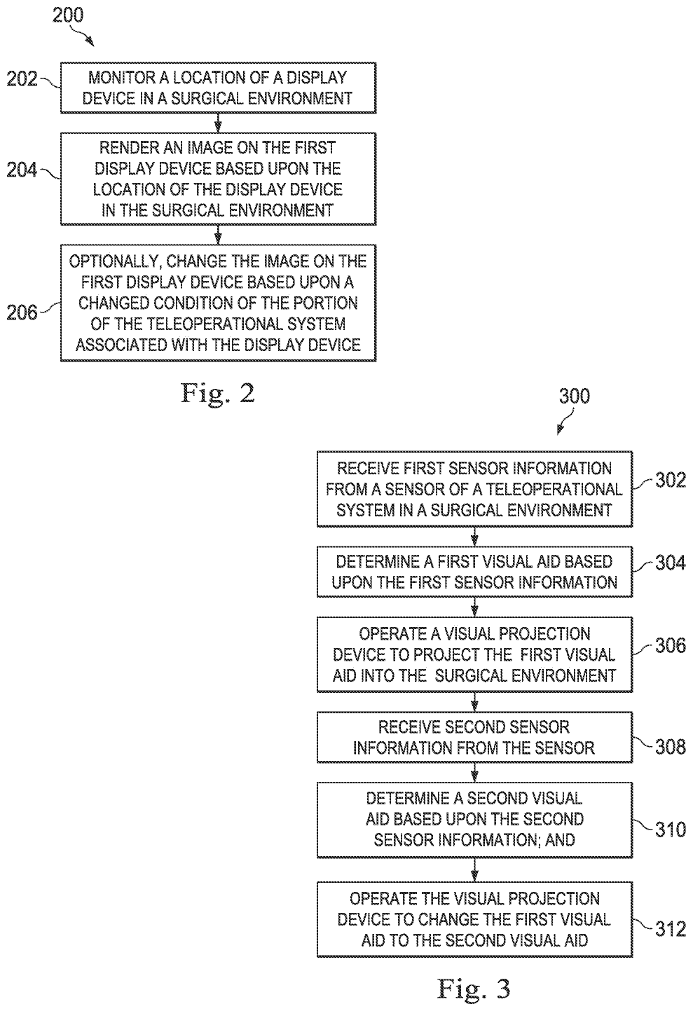

FIG. 2 illustrates a method 200 of providing information in a surgical environment (e.g. surgical environment 11). The method 200 is illustrated in FIG. 2 as a set of operations or processes 202 through 206. Not all of the illustrated processes 202 through 206 may be performed in all embodiments of method 200. Additionally, one or more processes that are not expressly illustrated in FIG. 2 may be included before, after, in between, or as part of the processes 202 through 206. In some embodiments, one or more of the processes may be implemented, at least in part, in the form of executable code stored on non-transitory, tangible, machine-readable media that when run by one or more processors (e.g., the processors of control system) may cause the one or more processors to perform one or more of the processes.

At a process 202, a location of a display device (e.g. the display 62a) is monitored or otherwise known in the surgical environment. The location of the display device may be determined in association with or relative to a teleoperational arm. For example, the display device 62 may be mounted to and fixed relative to the arm 54-1 such that the known kinematic position of the arm 54-1 provides the known location of the display device. If the arm 54-1 is moved, for example during a set-up procedure, the monitored change in the kinematic position of the arm 54-1 is used to determine the changed position of the display device. The position of the arm 54-1 may alternatively be determined by other types of sensors including electromagnetic position or optical sensors. Alternatively the location of the display device may be monitored by independent tracking of the display device using, for example, electromagnetic position sensors or optical sensors.

At a process 204, an image on the first display device is rendered based on the known or monitored location of the display device in the surgical environment. If the monitored location of the display device is on a teleoperational arm, the image may be associated with teleoperational arm or an instrument attached to that teleoperational arm. For example, if the teleoperational assembly 12 discovers a problem with one or more of the arms 54, the medical instrument systems 14, the endoscopic imaging system 15, and/or with other components, the teleoperational assembly 12 may operate the displays 62a-d located on the arms 54 experiencing problems to display information configured to facilitate resolution of said problems. Displays 62a-d may display a warning image, a diagnosis of the problem, a suggested resolution of the problem, written instructions for identifying and/or resolving the problem, an animation depicting the location and/or resolution of the problem, or some combination thereof. The images may display animations or instructions that reflect the current pose of the teleoperational arm as viewed from a clinician's perspective or a common perspective (e.g. from the front of the assembly 12) so that minimal user interpretation is required to understand the image.

For example, in the case that the surgical tool 30a has expired, the teleoperational assembly 12 may operate the display 62a on arm 54-1 to display a message indicating that the surgical tool 30a has expired and that a replacement should be installed. Providing alerts and instructions on the display 62a located on the arm 54-1 may increase the probability that operating room clinicians and/or maintenance workers will correctly identify the instrument to be replaced and expedite the resolution process. As another example, if a collision between two arms 54-1, 54-2 occurs or is anticipated by the control system 20, the display devices 62a and 62b may display guidance images. The images displayed on the display devices 62a and 62b may be different. For example the image on device 62a may provide instructions for moving arm 54-1 to prevent collision and the image on device 62b may provide different instructions for moving arm 54-2 to prevent collision. In some instances, it may be advantageous to exchange instruments between arms to resolve collisions. An animation that depicts exchanging instruments between the arms can be displayed concurrently on adjacent or non-adjacent arm displays to clarify the recommended exchange process. As another example, control signals between operator input system and the medical instrument system may be monitored to determine whether an instrument is currently grasping or applying force to tissue above a predetermined threshold level of force. If the instrument is currently grasping tissue, that status may be displayed on the respective arm display device. This may help improve troubleshooting and prevent tissue damage when bedside staff are correcting arm collisions or disengaging instruments.

The monitored location of the display device may additionally or alternatively provide the position of the display device in the surgical environment. That position of the display device may be compared to the positions of other tracked personnel or equipment in the surgical environment. Thus, a display device within a predetermined vicinity of a tracked clinician may be for selected for displaying guidance information for a tracked clinician. For example, the nearest display device may be used to provide training content based on the tracked clinicians skill level or experience. Displayed images may be presented from the vantage point of the tracked clinician.

At an optional process 206, the image on the display device changes based on a changed condition of the teleoperational arm to which the display device is attached. For example, the default image on the display device may be an arm or instrument status. The changeable condition may be an error status, an instrument expiration status, a collision status, a position, or another condition related to the state of the teleoperational arm or attached instrument. For example, during a manual teleoperational arm set-up procedure, the images on the display device 62a may change as the position of the arm 54-1 is adjusted to provide real-time guidance to the clinician adjusting the arms. The displayed images may portray the current pose of the arm and show how the arm should be manually repositioned. Additionally, the changeable condition may be an indication of arm activity and progress such as the progression of clamping or firing of a stapler. The changeable condition may also relate to the cable connections such as the sensed absence of an electrocautery cable.

In addition to identifying problems with arms 54, the displays 62 may display other information regarding the status of the arms 54. The displays 62 may be dynamically updated as the teleoperational assembly 12 continuously monitors the status of the arms 54. The displays 62 may display information about which tools or instruments are located on the arms 54, the number of times said tools or instruments have been used, the expiration date of said tools or instruments, other information, or combinations thereof. The displays 62 may display images of tools or instruments, animations of tools or instruments, descriptions of tools or instruments, usage graphs, usage timelines, other images, or combinations thereof.

The displays 62 may serve as input interfaces allowing clinicians to issue instructions to the teleoperational assembly 12. In various embodiments, the displays 62 may feature either capacitive or resistive touch screens and may comprise a Graphical User Interface (GUI). In some cases, a resistive touch screen may be desired. For example, a resistive touch screen may help preserve sterility by allowing a clinician to interact with the touch screen through use of a stylus or other instrument without having to touch the screen directly, which may ruin the sterility of the clinician's hand.

The options available to a clinician interacting with a display 62 may be variable depending on the tool in use by the arm on which the display 62 is mounted. For example, when the tool is a stapler, the clinician may be able to view the type of stapler reload installed, view the clamped status of the instrument, view maintenance reports, view the general status of the stapler, and/or order a reload of staples, among other things. By contrast, when the tool is an endoscope, the clinician may be able to view a images being captured by the endoscope, adjust the zoom of the endoscope, adjust the view orientation (e.g., angled up or down), order that a snapshot be taken, view maintenance reports, and/or view the status of the endoscope, among other things.

In various embodiments, the displays may be portable within the surgical environment. For example the displays may be tablets carried by a circulating clinician. The portable displays may provide context sensitive troubleshooting guidance that provides multiple levels of assistance. The assistance may include visual/animated content that are dependent on the state of the teleoperational system, a searchable electronic user manual, or messaging or two-way video calling. The display may also provide a barcode scanner for scanning medical equipment or instruments to receive further information.

FIG. 3 illustrates another method 300 of providing information in a surgical environment according to an embodiment of the disclosure. The method 300 illustrates the use of a projector (e.g., projector 60) to provide visual aids to a clinician in the surgical environment. The method 300 is illustrated in FIG. 3 as a set of operations or processes 302 through 312. Not all of the illustrated processes 302 through 312 may be performed in all embodiments of method 300. Additionally, one or more processes that are not expressly illustrated in FIG. 3 may be included before, after, in between, or as part of the processes 302 through 312. In some embodiments, one or more of the processes may be implemented, at least in part, in the form of executable code stored on non-transitory, tangible, machine-readable media that when run by one or more processors (e.g., the processors of control system) may cause the one or more processors to perform one or more of the processes. Prior to process 302, the system may recognize the docked status and control state of the teleoperational assembly 12 so that any displayed information is appropriate for the current docked status and control state.

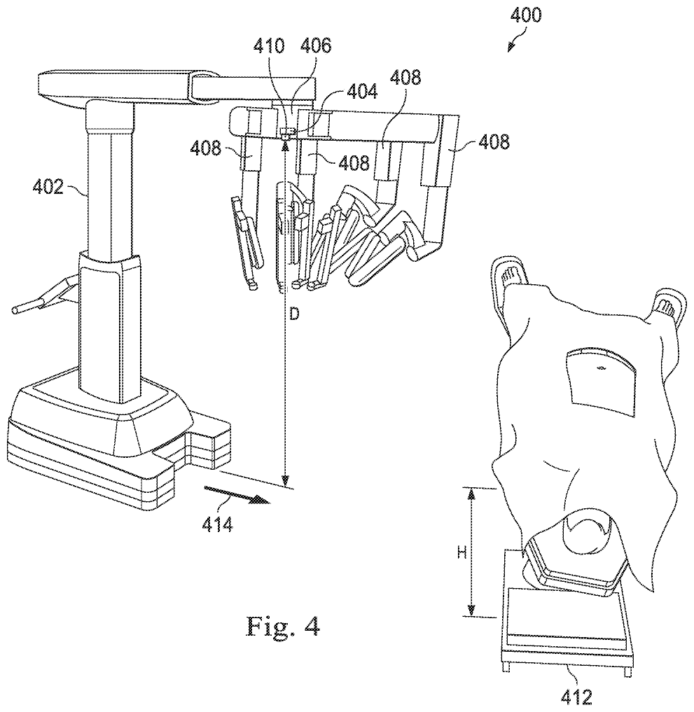

At a process 302, sensor information is received from a sensor (e.g., sensor 61) of a teleoperational system. At a process 304, a first visual aid is determined based on the sensor information. At a process 306, a visual projection device (e.g. projector 60) is operated to project the visual aid into the surgical environment. Processes 302-206 are further illustrated with reference to FIG. 4 which illustrates a surgical environment 400 including a teleoperational assembly 402 which may be substantially similar to assembly 12 and a projector 404 which may be substantially similar to projector 60. The projector 404 is coupled to an orienting platform 406 to which teleoperational arms 408 are coupled. A depth sensor 410 (e.g., sensor 61) measures a distance D downward into the work zone from the sensor to a obstructing surface. To determine the appropriate visual aid to project, the distance D is compared to a predetermined value associated with a height H of an operating table 412. If the distance D is approximately the same as the known height of the teleoperational assembly or is greater than the predetermined value, the sensor information indicates the absence of a patient or operating table in the work zone. Based on the absence of a patient in the work zone, a directional visual aid 414 such as an arrow is projected from the projector 404. The arrow is used during the initial approach of the patient and operating table to confirm the location of the center of the orienting platform 406. The direction of the arrow may provide a direction for delivering the orienting platform to the work zone. The orientation of the arrow may be determined to align with the base of the teleoperational assembly 402. The orientation of the arrow may be independent of the current orientation of the orienting platform.

Optionally, the teleoperational assembly 12 may be in wired or wireless communication with the operating table 412 such that the teleoperational assembly 12 is able to determine its position relative to the operating table. For example, the operating table and/or the teleoperational assembly 12 may include a wireless tracker such as a Global Positioning System (GPS) tracker. The teleoperational assembly 12 may receive ongoing positioning updates from the operating table. The positioning updates may be sent continuously, about every half second, about every second, about every three seconds, about every five seconds, about every 10 seconds, in response to a change in the position of the teleoperational assembly 12, in response to a request, in response to user instructions, at some other interval, or in response to some other stimulus.

The visual aid 414 may be projected downward onto the floor of the operating room or elsewhere. The aid 414 may be accompanied by audible cues such as tones, beeps, buzzes, an audio recording, other audible cues, or combinations thereof. In an alternative embodiment, visual aid may comprise a plurality of arrows aligned with the base of the assembly 402. In an alternative embodiment, the projected arrow may be adjusted in size, orientation, color, or another quality, in real time, as it receives updated positioning information from the operating table. In alternative embodiments, the visual aid may comprise an image of footprints, shoeprints, written cues, alphanumberic aids, a colored line, a footpath, stepping stones, a pointing finger, an outline of a human form, an animated image, or combinations thereof.

Referring again to FIG. 3, at a process 308, additional sensor information is received from the sensor. At a process 310, a visual aid is determined based on the additional sensor information. At a process 312, the visual projection device changes the visual aid of process 304 to the visual aid of process 310.

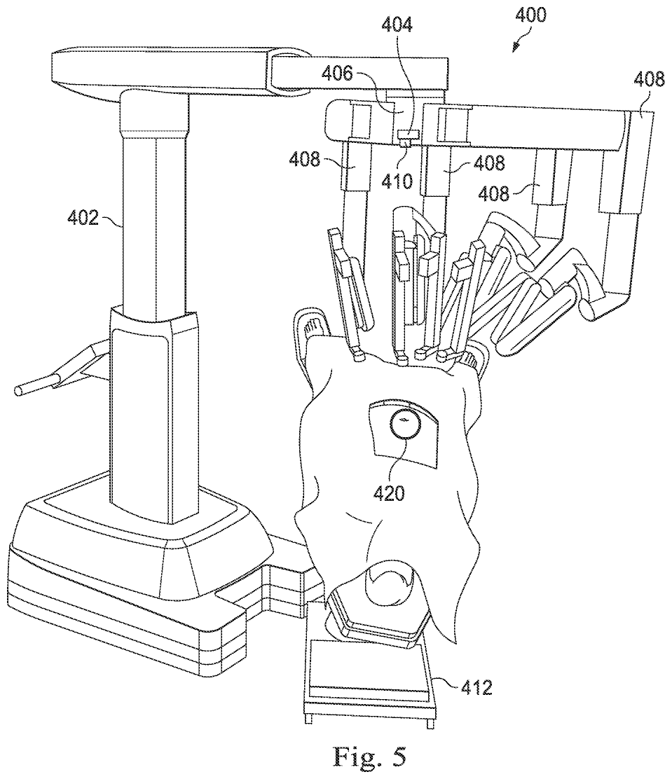

Processes 308-312 are further illustrated with reference to FIG. 5. When the sensor 410 determines that a patient has moved into the work zone of the teleoperational assembly or the teleoperational system has otherwise entered an orienting platform positioning mode, the directional visual aid 414 is replaced with a visual aid 420 to assist with the next step of the set-up process. In this embodiment, the visual aid 420 is an orienting platform positioning aid which may be a circle projected onto the patient. The circle is used to position the orienting platform 406 over the patient. The circle is adaptively sized by the projector 404 to appear with a fixed radius, independent of the distance between the sensor 410 and the patient. The projected radius may be invariant to the distance between the projector and the patient. The sensor may be used to determine the projection distance to then compute the appropriate radius to be projected. The fixed radius may be based on the positioning tolerance of the orienting platform for docking the teleoperational arms to cannula positioned in patient incisions. In one example embodiment, the circle may have a radius of approximately 3 inches. Various symbols may be used as an orienting platform positioning aid with the circle being particularly suitable because it does not imply an orientation direction which may be unnecessary during platform positioning. In addition to or as an alternative to the circle, the visual aid may comprise an image of a crosshairs, an ‘X’, a target featuring concentric rings, a square, a rectangle, a smiley face, an outline of a human form, an animated image, or combinations thereof. In an alternative embodiment, an optical element such as a Fresnel lens may be used in front of the projector to achieve an orthographic projection such that the projected visual does not change size as the height of the orienting platform changes.

The desired precision in positioning the orienting platform 406 prior to surgery may be variable depending on the procedure to be performed and/or on the physical characteristics of the patient. As discussed above, the database 27 may comprise a list of patient profiles and a list of procedures to be performed on said patients. Accordingly, the teleoperational assembly 12 may determine the procedure to be performed on the patient currently on the operating table and determine physical characteristics of said patient based on information contained in the database 27 and may adjust the visual aid based on one or the other or both of these determinations.

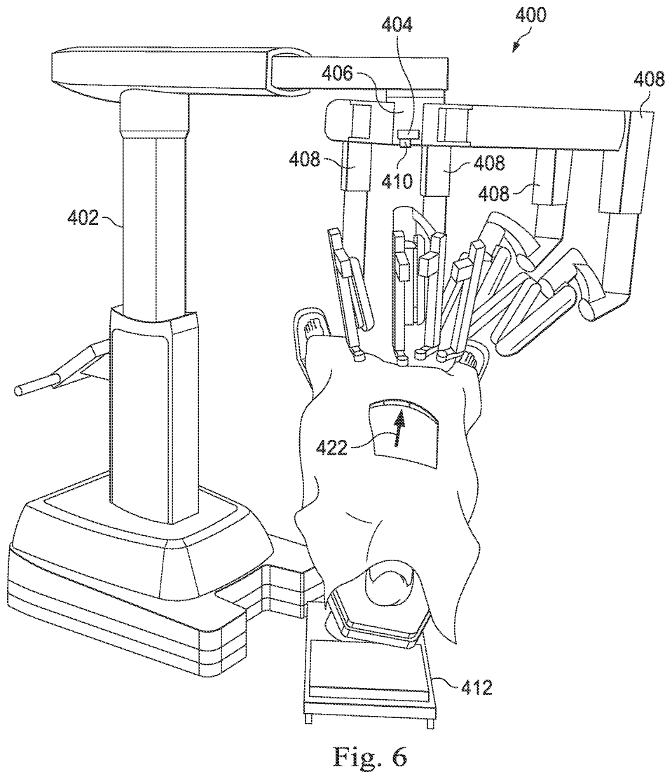

Processes 308-312 are further illustrated with reference to FIG. 6 in which the orienting platform positioning aid 420 is replaced with an orienting platform orientation aid 422. When the sensor 410 determines that a patient has been properly positioned in the work zone or the teleoperational system has otherwise entered an orienting platform orientation mode, the directional visual aid 414 is replaced with a visual aid 420 to assist with the next step of the set-up process. In this embodiment the orientation platform orientation aid 422 is a linear arrow indicating the working direction of the teleoperational arms. The aid 422 may minimize confusion of the principal working direction of the orienting platform 406 and the arms 408. The arms 408 operate in a pitched forward orientation. Providing the aid 422 guides the set-up operator to position the orienting platform and the arms so that the pitch of the arms is in the direction of the aid.

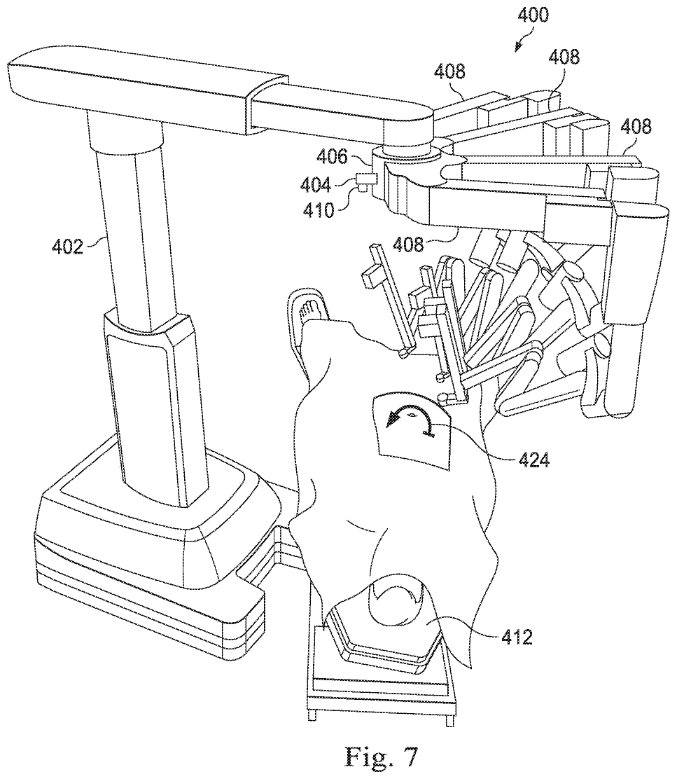

FIG. 7 illustrates an orienting platform orientation aid 424 which in this embodiment is a curved arrow displayed when the orienting platform 406 is approaching a rotational limit. The orienting platform range of motion may be limited to +/−180 degrees of rotation. If sensor information indicates that a user is attempting to rotate the orienting platform beyond the 180 degree range, the aid 424 may be projected to alert the user to the need to rotate the orienting platform in the opposite direction.

After the teleoperational assembly is satisfactorily positioned and oriented, the surgeon may begin the scheduled surgery. The projector may provide one or more visual aids during surgery. The visual aids may be based on information contained in the database 27 and/or based on information received by the teleoperational assembly 12 during surgery, e.g., from the endoscopic imaging system 15. For example, the projector may project an image suggesting an incision site onto a patient or elsewhere based on the procedure to be performed. For further example, the projector may project preoperative images of internal anatomic structures onto a patient or elsewhere based on information received from the endoscopic imaging system 15.

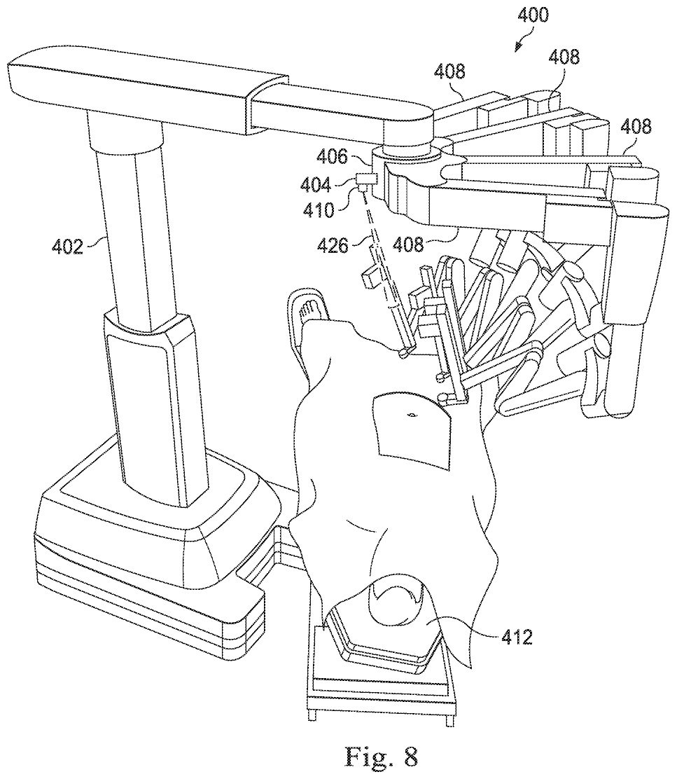

FIG. 8 illustrates a highlighting visual aid 426. When sensor information indicates that attention is needed at a particular location in the work zone, the projector 404 projects visual aid 426 onto the location that requires attention. The content of the visual aid 426 may be constant or modulated light, symbols, alphanumeric content or other visual content to draw attention to highlighted area. For example the visual aid 426 may appear on the patient, on an instrument, on an arm, on a location of arm collision, or another location in the work zone. In one example, the visual aid may be a spotlight used to highlight the position of interference or collision between one or more arms. Alternatively, the visual aid may comprise a depiction of the arms in contact with each other, a written warning of the contact, other images, or combinations thereof. In another example, the visual aid may be information about an error state of the highlighted instrument. In another embodiment, the color or content of the visual aid may change as an arm is manually moved toward an optimal pose. For example, as an arm is being moved toward the proper position for surgery, the projector may generate a visual aid indicating that the arm is getting closer to the proper position. The visual aid may be a light projected onto the arm being moved such that the light becomes increasingly green as the arm 54 gets closer to the proper position and becomes increasingly red as the arm gets farther away from the proper position. Any other colors may be additionally or alternatively used. A strobe, spotlight, or other cue may be generated when the arm has reached the proper position.

The teleoperational assembly 12 may be configured to monitor the condition of its various components, including medical instrument systems 14, endoscopic imaging system 15, and arms 54, and to identify maintenance problems. The projector may generate one or more visual aids to facilitate resolution of such problems. For example one maintenance problem that may be encountered is the failure or expiration of a tool such as one of the surgical instruments. The projector may highlight the failed or expired tool as discussed above and/or may project an image identifying the failed or expired tool onto the patient or elsewhere. The image identifying the failed or expired tool may comprise a depiction of the failed or expired tool, a depiction of the arm on which the failed or expired tool is located, a written warning of the failure or expiration, a spotlight, an animated image, other images, or combinations thereof. A projected spotlight may also highlight the portion of the instrument housing where the clinician will need to insert an accessory to manually open the instrument jaws prior to removal.

The visual aids generated by the projector may be variable depending on the experience level of the clinicians in the operating room. For example, additional or more detailed visual aids may be given when the clinicians in the operating room have a low level of experience. In an embodiment, the experience level of clinicians in the operating room for visual aid determination purposes may be limited to that of the least experienced clinician. Alternatively, the experience level of clinicians in the operating room for visual aid determination purposes may be the average experience level or that of the most experienced clinician. In some cases, the surgeon may be exempted from the calculation of experience level.

In various embodiments, the images and information displayed to the user may provide safety related guidance. For example, the information may guide a user through solutions including in cases of power failure. Manipulator mounted displays may include battery back-up and high availability isolation to provide instructions for safe egress of instruments from the patient anatomy in the event of power loss or a non-recoverable system failure. In another example, if a manipulator arm becomes inoperable, a manipulator mounted display may provide information for correctly positioning the arm out of the way of other arms or other components of the teleoperational assembly.

In various embodiments, the images and information displayed to the user may provide information about system interruptions related to expired tools, invalid tools, and energy instrument cable connection status. In various embodiments, the images and information displayed to the user may provide information about instrument type, usage life remaining on an instrument, endoscope status, manipulator arm status (e.g., in progress, waiting on input), instrument state (e.g., grip, stapler clamp, busy), dual console and single site clarity (e.g., depiction associating each instrument to one of the surgeon consoles, depiction of left/right hand association), a manipulator numerical identifier, an undocked manipulator arm, managing and avoiding collisions, and proper manipulator arm stowage guidance. In various embodiments, the images and information displayed to the user may provide guided tutorials. Such tutorials may be provided in response to a user request for help or may be provided in a training mode of the system. In various embodiments, the images and information displayed to the user may optionally provide optimization information regarding, for example, flex position or patient clearance. Other customized information may also be displayed.

One or more elements in embodiments of the invention may be implemented in software to execute on a processor of a computer system such as control processing system. When implemented in software, the elements of the embodiments of the invention are essentially the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device, The code segments may be downloaded via computer networks such as the Internet, Intranet, etc.

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the embodiments of the invention are not described with reference to any particular programming language. It will be appreciated that a variety of programming languages may be used to implement the teachings of the invention as described herein.

While certain exemplary embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art.

1-47. (canceled)

48. A teleoperational system in a surgical environment comprising:
a teleoperational assembly including a first teleoperational arm;
a visual projection device coupled to the teleoperational assembly;
a sensor; and
one or more processors configured to:
receive first sensor information from the sensor;
determine a first visual aid based upon the first sensor information;
operate the visual projection device to project the first visual aid into the surgical environment; and
operate the visual projection device to change the first visual aid to a second visual aid based on second sensor information received from the sensor.

49. The teleoperational system of claim 48 wherein the sensor is a depth sensor.

50. The teleoperational system of claim 48 wherein the first sensor information indicates an absence of an obstruction in a work zone of the teleoperational assembly.

51. The teleoperational system of claim 50 wherein the first visual aid is a direction indicator for guidance of the teleoperational assembly into the work zone.

52. The teleoperational system of claim 51 wherein the second sensor information indicates a presence of an operating table in the work zone and the second visual aid is a teleoperational assembly positioning indicator.

53. The teleoperational system of claim 48 wherein at least one of a size of the second visual aid relative to an operating table or an orientation of the second visual aid relative to the operating table remains unchanged as the visual projection device moves with the teleoperational assembly.

54. The teleoperational system of claim 48 wherein the teleoperational assembly includes an orienting platform to which the first teleoperational arm is coupled and wherein the second sensor information indicates selection of an orienting platform orientation mode, and wherein the second visual aid is an orienting platform orientation indicator.

55. The teleoperational system of claim 48 wherein the second sensor information indicates a system error and wherein the second visual aid includes a warning or instructions for correcting the system error.

56. The teleoperational system of claim 48 wherein the visual projection device is coupled to the first teleoperational arm and wherein the one or more processors are further configured to:
determine a change in a condition associated with the first teleoperational arm; and
operate the visual projection device based on the changed condition.

57. The teleoperational system of claim 48 wherein responsive to the first sensor information indicating information about the first teleoperational arm, the first visual aid is directed toward the first teleoperational arm.

58. The teleoperational system of claim 48 wherein the teleoperational assembly includes an orienting platform to which the first teleoperational arm is coupled and wherein an appearance of the first visual aid remains unchanged when the orienting platform is rotated.

59. A method comprising:
receiving first sensor information from a sensor of a teleoperational system in a surgical environment;
determining a first visual aid based upon the first sensor information;
operating a visual projection device to project the first visual aid into the surgical environment, wherein the visual projection device is coupled to a teleoperational assembly of the teleoperational system;
receiving second sensor information from the sensor;
determining a second visual aid based upon the second sensor information; and
operating the visual projection device to change the first visual aid to the second visual aid.

60. The method of claim 59 wherein the first sensor information is a first distance measurement from the sensor.

61. The method of claim 60 wherein the first distance measurement exceeds a predetermined value, indicating an absence of an obstruction in a work zone of the teleoperational assembly.

62. The method of claim 61 wherein the first visual aid is a direction indicator for guiding the teleoperational system into the work zone.

63. The method of claim 62 wherein the second sensor information is a second distance measurement from the sensor that is less than the predetermined value, indicating a presence of an operating table in the work zone and wherein the second visual aid is a teleoperational assembly positioning indicator.

64. The method of claim 59 wherein a size of the second visual aid relative to an operating table remains unchanged as the visual projection device moves with the teleoperational assembly.

65. The method of claim 59 wherein an orientation of the second visual aid relative to an operating table remains unchanged as the visual projection device moves with the teleoperational assembly.

66. The method of claim 59 further comprising:
responsive to the first sensor information indicating information about a first teleoperational arm of the teleoperational assembly, projecting the first visual aid toward the first teleoperational arm.

67. The method of claim 66 further comprising:
responsive to the second sensor information indicating information about a second teleoperational arm of the teleoperational assembly, projecting the second visual aid toward the second teleoperational arm.
